(19) United States
(12) Patent Application Publication
(10) Pub. No.: US 2013/0153209 A1
(43) Pub. Date: Jun. 20, 2013

(54) SHEWANELLA ENRICHMENT FROM OIL RESERVOIR FLUIDS

(71) Applicants: Edwin Hendrickson, Hockessin, DE (US); Abigail Luckring, West Chester, PA (US)

(72) Inventors: Edwin Hendrickson, Hockessin, DE (US); Abigail Luckring, West Chester, PA (US)

(21) Appl. No.: 13/713,064

(22) Filed: Dec. 13, 2012

Related U.S. Application Data
(60) Provisional application No. 61/570,553, filed on Dec. 14, 2011.

(51) Int. Cl. C12N 1/20; E21B 43/00

(52) U.S. Cl. CPC C12N 1/20; E21B 43/00
USPC 166/246; 435/252.1; 435/6.11

(57) ABSTRACT

Methods for enriching microbial populations that are indigenous to oil reservoir fluids to obtain Shewanella-enriched populations and isolated Shewanella strains useful for enhancing oil recovery from an oil reservoir are presented. In addition, the enriched populations may contain bacteria belonging to the Arcobacter genus.

### CROSS REFERENCE TO RELATED APPLICATIONS

This application claims priority from U.S. Provisional Application No. 61/570,553, filed Dec. 14, 2011, which is herein incorporated by reference in its entirety.

### FIELD OF THE INVENTION

This disclosure relates to the field of environmental microbiology. More specifically, methods for enriching microbial populations that are indigenous to oil reservoir fluids to obtain Shewanella-enriched populations and isolated Shewanella strains useful for enhancing oil recovery from an oil reservoir are presented herein.

### BACKGROUND OF THE INVENTION

During recovery of oil from oil reservoirs, typically only a minor portion of the original oil in the oil-bearing strata is recovered by primary recovery methods, which uses the natural forces present in an oil reservoir. To improve oil recovery, a variety of supplemental recovery techniques, such as water flooding which involves injection of water through well bores into the oil reservoir, have been used. As water moves into the reservoir from an injection well and moves through the reservoir strata, it displaces oil to one or more production wells where the oil is recovered. One problem commonly encountered with water flooding operations is poor sweep efficiency of injection water. Poor sweep efficiency occurs when water preferentially channels through highly permeable zones of the oil reservoir as it travels from the injection well(s) to the production well(s), thus bypassing less permeable oil-bearing strata. Oil in the less permeable zones is thus not recovered.

Recovery of oil from subterranean formations may be enhanced by the effects of microorganisms that have characteristics such as promoting oil release by reduction in surface and interfacial tensions and/or forming bio-plugs to reduce channeling to improve sweep efficiency. One approach for Microbial Enhanced Oil Recovery (MEOR) is to stimulate growth of microorganisms having these properties that are indigenous to or inoculated into subterranean formations of oil reservoirs. Processes for promoting growth of indigenous microbes by injecting nutrient solutions into subterranean formations are disclosed in U.S. Pat. No. 4,558,739 and U.S. Pat. No. 5,083,611. Microorganisms belonging to the genus Shewanella were isolated from oil reservoir samples using media containing nitrate or Fe(III) as the electron acceptor and were found to be useful for MEOR processes as disclosed in U.S. Pat. No. 7,776,795 and US 2011-0030956. Microorganisms belonging to the genus Arcobacter were also isolated and found to be useful for MEOR processes as disclosed in commonly owned and co-pending U.S. patent application Ser. No. 13/280,972.

There remains a need for methods of obtaining targeted populations of microorganisms or isolated microorganisms from oil reservoir fluids that include microorganisms belonging to the Shewanella and Arcobacter genera, since these microorganisms are effective in MEOR processes.

### SUMMARY OF THE INVENTION

The invention relates to methods of enrichment for microorganisms belonging to the Shewanella genus, as well as to methods of isolating microorganisms belonging to the Shewanella genus by growth in media containing specified types of electron acceptors. In addition, salt concentrations may be specified.

Accordingly, the invention provides enriching a microbial population comprising:
a) providing an environmental sample from an oil reservoir fluid comprising an indigenous microbial population; and
b) growing said sample using medium comprising salt, at least one carbon source, and an electron acceptor selected from organic electron acceptors and metal ion electron acceptors to obtain an enriched microbial population;
wherein the proportion of Shewanella cells in the enriched microbial population of (b) is greater than the proportion of Shewanella cells in the environmental sample of (a).

In another embodiment the invention provides a method for isolating a strain of the Shewanella genus comprising:
a) providing an environmental sample from an oil reservoir fluid comprising an indigenous microbial population;
b) growing said sample using medium comprising salt, at least one carbon source, and an electron acceptor selected from organic electron acceptors and metal ion electron acceptors to obtain an enriched microbial population;
c) growing the enriched microbial population of (b) on medium having salt concentration that is between about 10 ppt and 40 ppt to obtain isolated colonies; and
d) screening the isolated colonies of (c) by rDNA sequence analysis against known microbial rDNA sequences;
wherein at least one Shewanella strain is identified.

In yet another embodiment the invention provides a method for enhancing oil recovery from an oil reservoir comprising:
a) providing an enriched microbial population obtained by the method of claim 1 or 3;
b) introducing the enriched microbial population of (a) into an oil reservoir;
c) introducing a nutrient solution comprising at least one carbon source and at least one electron acceptor into said oil reservoir; and
d) recovering oil from the oil reservoir;
wherein growth of the enriched microbial population enhances oil recovery.

### BRIEF DESCRIPTION OF SEQUENCES

The invention can be more fully understood from the following detailed description and the accompanying sequence descriptions, which form a part of this application.

The following sequences conform with 37 C.F.R. §§1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (2009) and the sequence listing requirements of the EPO and PCT (Rules 5.2 and 49.5(a-bis), and Section 208 and Annex C of the Administrative Instructions. The symbols and format used for nucleotide and amino acid sequence data comply with the rules set forth in 37 C.F.R. §1.822.

SEQ ID NO:1 is the nucleotide sequence of reverse PCR primer 1492R.

SEQ ID NO:2 is the nucleotide sequence of forward PCR primer 8F.

SEQ ID NO:3 is the nucleotide sequence of the 16S rDNA used to identify the Shewanella homology cluster.

SEQ ID NO:4 is the nucleotide sequence of the 16S rDNA used to identify the Arcobacter homology cluster.

### DETAILED DESCRIPTION

Applicants specifically incorporate the entire content of all cited references in this disclosure. Unless stated otherwise, all percentages, parts, ratios, etc., are by weight. Trademarks are shown in upper case. Further, when an amount, concentration, or other value or parameter is given as either a range, preferred range or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

The invention relates to methods of obtaining from oil reservoir fluids, microbial consortia that have increased proportion of microorganisms belonging to Shewanella species with respect to the proportion of Shewanella in the original fluids. In addition, methods for isolating strains of Shewanella are disclosed. The obtained consortia or isolated strains may be used to increase oil recovery.

The following definitions are provided for the special terms and abbreviations used in this application:

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

The term “invention” or “present invention” as used herein is a non-limiting term and is not intended to refer to any single embodiment of the particular invention but encompasses all possible embodiments as described in the specification and the claims.

As used herein, the term “about” modifying the quantity of an ingredient or reactant of the invention employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities. In one embodiment, the term “about” means within 10% of the reported numerical value, preferably within 5% of the reported numerical value.

“Petroleum” or “oil” is a naturally occurring, flammable liquid found in rock and sand formations in the Earth, which consisting of a complex mixture of hydrocarbons and polycyclic aromatic hydrocarbon of various molecular weights, plus other organic compounds.

The terms “oil reservoir” and “oil-bearing stratum” may be used herein interchangeably and refer to a subterranean or sub sea-bed formation from which oil may be recovered. The formation is generally a body of rocks, consolidated sand and soil having sufficient porosity and permeability to store and transmit oil.

The term “well bore” refers to a channel from the surface to an oil-bearing stratum with enough size to allow for the pumping of fluids either from the surface into the oil-bearing stratum (injection well) or from the oil-bearing stratum to the surface (production well).

The term “Microbial Enhanced Oil Recovery” (MEOR) is a biological based technology consisting in modifying microbial function or structure, or both, of microbial environments or microbes, or both existing in oil reservoirs. The ultimate aim of MEOR is to improve the recovery of oil entrapped in porous media. MEOR is a tertiary oil extraction technology allowing the partial recovery of residual of oil in effect, increasing the life of oil reservoirs.

The term “remediation” refers to the process used to remove hydrocarbon contaminants from contaminant-altered environment.

The term “bioremediation” refers to the use of microorganisms to remediate or detoxify contaminants form a contaminant-altered environment

The term “electron donor” refers to a molecular compound that gives or donates an electron(s) during cellular respiration.

The term “electron acceptor” refers to a molecular compound that receives or accepts an electron(s) during cellular respiration. Microorganisms obtain energy to grow by transferring electrons from an “electron donor” to an “electron acceptor”. During this process, the electron acceptor is reduced and the electron donor is oxidized. Examples of electron acceptors include oxygen, nitrate, fumarate, iron (III), manganese (IV), sulfate and carbon dioxide. Sugars, low molecular weight organic acids, carbohydrates, fatty acids, hydrogen and crude oil or its components such as petroleum hydrocarbons or polycyclic aromatic hydrocarbons are examples of compounds that can act as electron donors. The terms “denitrifying” and “denitrification” mean reducing nitrate or nitrite for use in respiratory energy generation.

“Adhered to” refers to coating or adsorption of a liquid to a solid surface of at least 10% areal coverage.

The term “wetting” refers to the ability of a liquid to maintain contact with a solid surface, resulting from intermolecular interactions when the two are brought together. The degree of wetting (expressed as “wettability”) is determined by a force balance between adhesive and cohesive forces.

“Wetting agent” refers to a chemical such as a surfactant that increases the water wettability of a solid or porous surface by changing the hydrophobic surface into one that is more hydrophilic. Wetting agents help spread the wetting phase (e.g., water) onto the surface thereby making the surface more water wet.

“Interface” as used herein refers to the surface of contact between a water layer and an oil layer, a water layer and a solid surface layer, and an oil layer and a solid surface layer.

“Hydrocarbon-coated” as used herein refers to a coating of a hydrocarbon to a solid surface of at least 10% areal coverage.

The term “components of a subsurface formation” refers to rock, soil, brine, sand, clay or mixtures thereof of either subterranean or seabed formations, that have come in contact with one or more hydrocarbon. These components may be part of an oil well or reservoir. At least a portion of the components include some hydrocarbon-coated surfaces, including particles with coated surfaces.

“Wettability” refers to the preference of a solid to contact one liquid, known as the wetting phase, rather than another. Solid surfaces can be water wet, oil wet or intermediate wet. “Water wettability” pertains to the adhesion of water to the surface of a solid. In water-wet conditions, a thin film of water coats the solid surface, a condition that is desirable for efficient oil transport.

The term “adhesive forces” refers to the forces between a liquid and solid that cause a liquid drop to spread across the surface.

The “cohesive forces” refers to forces within the liquid that cause the drop to ball up and avoid contact with the surface.

The term “water flooding” refers to injecting water through well bores into an oil reservoir. Water flooding (secondary oil recovery) is performed to flush out oil from an oil reservoir when the oil no longer flows on its own out of the reservoir.

The term “sweep efficiency” relates to the fraction of an oil-bearing stratum that has seen fluid or water passing through it to move oil to production wells during water flooding. One problem that can be encountered with water flooding operations is the relatively poor sweep efficiency of the water, i.e., the water can channel through certain portions of a reservoir as it travels from injection well(s) to production well(s), thereby bypassing other portions of the reservoir. Poor sweep efficiency may be due, for example, to differences in the mobility of the water versus that of the oil, and permeability variations within the reservoir which encourage flow through some portions of the reservoir and not others.

The term “injection water” refers to fluid injected into oil reservoirs for secondary oil recovery. Injection water may be supplied from any suitable source, and may include, for example, sea water, brine, production water, water recovered from an underground aquifer, including those aquifers in contact with the oil, or surface water from a stream, river, pond or lake. As is known in the art, it may be necessary to remove particulate matter including dust, bits of rock or sand and corrosion by-products such as rust from the water prior to injection into the one or more well bores. Methods to remove such particulate matter include filtration, sedimentation and centrifugation.

The term “production water” means water recovered from production fluids extracted from an oil reservoir. The production fluids contain both natural water associated with the reservoir and/or water used in secondary oil recovery, and crude oil produced from the oil reservoir.

The term “biofilm” means a film or “biomass layer” of microorganisms. Biofilms are often embedded in extracellular polymers, which adhere to surfaces submerged in, or subjected to, aquatic environments. Biofilms consist of a matrix of a compact mass of microorganisms with structural heterogeneity, which may have genetic diversity, complex community interactions, and an extracellular matrix of polymeric substances.

The term “plugging biofilm” means a biofilm that is able to alter the permeability of a porous material, and thus retard the movement of a fluid through a porous material that is associated with the biofilm. The term “simple nitrates” and “simple nitrites” refer to nitrate (NO₃) and nitrite (NO₂), respectively.

The term “bioplugging” refers to making permeable material less permeable due to the biological activity, particularly by a microorganism.

The term “indigenous microorganisms” refers to the microorganisms that are native to the oil reservoir fluids and subterranean matrices.

The term “inoculated microorganisms” refers to the microorganism that are introduced to the oil reservoir fluids and subterranean matrices by injecting the microbes through a well bore into the oil reservoir substructure.

“Shewanella species” or “Shewanella spp.” refers to microorganisms phylogenetically classified by rDNA typing to the Shewanella genus. Members to Shewanella are Gram negative, metal-reducing, gamma-proteobacteria that are capable of reducing a wide range of terminal electron acceptors. These microorganisms gain energy to support anaerobic growth by coupling the oxidation of H₂ or organic matter to the redox transformation of a variety of multivalent metals, which leads to the precipitation, transformation, or dissolution of minerals.

The term “salt” includes any ionic compound that can create ions in water including, but not limited to KCl, SrCl, NaBr, NaCl, CaCl₂, and MgCl₂.

Obtaining Shewanella-Enriched Populations from Oil Reservoir Fluids

Disclosed herein are methods for increasing the proportion of microorganisms belonging to the Shewanella genus among total populations of indigenous microorganisms of oil reservoir fluids. Strains of Shewanella are desired for their properties that are useful for enhancing oil recovery, as described below. Oil reservoir fluids (i.e., an “environmental sample”) may be production waters or injection waters and contain heterogeneous mixtures of microorganisms. The genera to which these microorganisms belong can be identified by 16S ribosomal DNA sequence analysis, and the proportion of microorganisms in each genera in the population of an oil reservoir fluid sample can be determined, as described in the Examples herein.

Oil reservoir fluid samples which provide the environmental samples evaluated herein were from reservoirs in the Senlac field located in the province of Saskatchewan, Canada, and in the Wainwright field in the province of Alberta, Canada. The waters of these reservoirs have high salinity: in the range of 30 to 38 ppt and in the range of 60 to 68 ppt, respectively. Analysis herein, in Examples 1 and 2, of the microorganisms indigenous to these oil reservoir fluid samples detected no microorganisms belonging to the Shewanella genus. Methods were developed involving growing indigenous populations of oil reservoir fluid samples in media containing specified electron acceptors whereby microorganisms belonging to the Shewanella genus became detectable as a proportion of the population. These methods therefore enrich for Shewanella microorganisms in a microbial population.

In the present methods, microorganisms in a sample from an oil reservoir are grown using medium containing salt, at least one carbon source, and an electron acceptor selected from organic electron acceptors and metal ion electron acceptors. In addition, the medium contains other components to support microorganism growth which may include components such as vitamins, trace metals, nitrogen, phosphorus, magnesium, calcium, and/or buffering chemicals. The carbon source may be complex organic matter such as peptone, corn steep liquor, or yeast extract. In another embodiment the carbon source is a simple compound such as citrate, fumarate, maleate, butyrate, pyruvate, succinate, acetate, formate or lactate.

Organic electron acceptors that may be used include compounds such as fumarate, trimethylamine-N-oxide, dimethyl sulfoxide, pyruvate, glycine, and mixtures of these compounds. Fumarate may be in the form of any salt of fumaric acid, or fumaric acid itself may in this context be included in the term fumarate. Salts of fumarate may include a monosodium or disodium salt, calcium salt, magnesium salt, ammonium or diammonium salt, potassium or dipotassium salt, hydrochloride salt, or hydrated forms of any fumarate acid salt. In one embodiment the composition contains disodium fumarate (DSF).

Metal ion electron acceptors that may be used include compounds such as Fe(III), Mn(IV), Cr(IV), and mixtures of these compounds.

With growth of indigenous oil reservoir fluid populations of microorganisms using media containing these electron acceptors, the proportion of the population that belongs to the Shewanella genus (which are Shewanella cells) increases, in contrast to growth in media containing nitrate as the electron acceptor. The proportion of the population that belongs to the Shewanella genus increases to at least about 1%. The increase in proportion of Shewanella cells depends upon factors such as the distribution of microorganisms in the population indigenous to the fluid sample, the specific electron acceptor used, and the salt concentration of the medium. The proportion of the population that belongs to the Shewanella genus may increase to at least about 1%, 2%, 5%, 10%, 15%, 20%, 25%, 30%, 35%, 40%, 45%, 50%, or higher, including to at least about 90%.

The salt concentration of the medium may be adjusted to be favorable for the growth of microorganisms belonging to the Shewanella genus. As found in Example 3 herein, Shewanella microorganisms grow in media having a wide range of salt concentrations including low salt concentration (11 ppt) or high salt concentration (60 ppt), but grow best in salt concentrations of about 15-20 ppt. In one embodiment of the present method the salt concentration is between about 10 and 55 ppt. In other embodiments the salt concentration is between about 10 and 40 ppt, or about 15 and 35 ppt.

When using the present method, the enriched microorganism population may contain microorganisms belonging to the Arcobacter genus. The proportion of the population that belongs to the Arcobacter genus depends upon factors such as the distribution of microorganisms in the population indigenous to the fluid sample, the specific electron acceptor used, and the salt concentration of the medium. In particular, with use of an organic electron acceptor a large proportion of the population belong to the Arcobacter genus, such as greater than 20%. The salt concentration of the medium may be adjusted to be favorable for the growth of microorganisms belonging to the Arcobacter and Shewanella genera. For example, Arcobacter species will be reduced and may not be detected when using lower salt concentration such as about 34 ppt. Salt concentrations that are at least about 40 ppt may be used to obtain populations containing detectable levels of Arcobacter cells. In one embodiment the salt concentration is between about 40 and 55 ppt and Shewanella and Arcobacter cells are present in the population obtained. In another embodiment the salt concentration is at least about 50 ppt and the proportion of Arcobacter cells is at least about 20%.

### Isolating Shewanella Strains

Strains of microorganisms belonging to the Shewanella genus may be isolated from populations enriched for Shewanella cells that are obtained following the present methods as described above. Following the enrichment, samples are grown on medium having salt concentration that is between about 10 ppt and 40 ppt to isolate single colonies from which strains are grown. The 16S ribosomal DNA (rDNA) sequence is obtained for an isolated strain and compared to sequences in the NCBI rDNA database (˜260,000 rDNA sequences) using the BLAST algorithm (Altschul et al., Journal of Molecular Biology, 1990). A strain is identified as a Shewanella strain when the highest scoring sequence identity hit is from a known species of Shewanella.

The present method of enrichment using an organic or metal ion electron acceptor and growth on medium having a salt concentration supportive for growth of Shewanella cells allows isolation of Shewanella strains at high frequency. Of analyzed strains, at least one in ten, or nine, or eight, or seven, or six, or five, or four, or three, or two, or one, strains analyzed is a Shewanella strain.

### Use of Shewanella and Arcobacter Cells for MEOR

The present Shewanella-enriched microbial populations are useful for enhancing oil recovery. Shewanella cells have been shown to have properties that are beneficial for enhancing oil recovery from oil reservoirs. For example, disclosed in U.S. Pat. No. 7,776,795, which is incorporated herein by reference, is isolation of Shewanella putrefaciens strain LH4:18 (ATCC No. PTA-8822) from water samples from production and injection wells from Alaska North Slope oil fields by growth on distilled crude oil in medium containing nitrate and salts in a column reactor. This strain was shown to enhance oil release from sand. Additionally disclosed in US 2011-0030956, which is incorporated herein by reference, is the ability of Shewanella cells or materials or biomolecules produced by Shewanella to alter the wettability of a hydrocarbon coated surface, thereby releasing oil from the surface. Oil released in this manner from surfaces in an oil reservoir can be recovered in secondary oil recovery methods such as water flooding.

In some embodiments, in addition to Shewanella cells, the present enriched microbial populations contain Arcobacter cells. Arcobacter cells have been shown to have properties that are beneficial for enhancing oil recovery from oil reservoirs. As disclosed in commonly owned and co-pending U.S. patent application Ser. No. 13/280,972, which is incorporated herein by reference, Arcobacter strains including 97AE3-3 (ATCC No. PTA-11410) and 97AE3-12 (ATCC No. PTA-11409) isolated therein are able to form plugging biofilms. Strain 97AE3-12 grew in the presence of petroleum oil, in both low (15 ppt) and high salinity (64 ppt) denitrifying conditions. Plugging biofilms were produced in low (15 ppt) and high (35 ppt and 68 ppt) salinity media. Plugging biofilms were formed with either batch or continuous nutrient feeding. In addition, silica particle aggregation was demonstrated in high salinity (64 ppt) media. These properties demonstrate use of the Arcobacter strains for forming biofilms to plug highly permeable zones in permeable sand or rock of oil reservoirs. Plugging of hyperpermeable zones may reroute water towards less permeable, more oil rich areas thereby enhancing sweep efficiency leading to increased oil recovery.

### Methods of Enhancing Oil Recovery

The present Shewanella-enriched microbial populations, in some embodiments additionally containing Arcobacter cells, may be introduced into an oil reservoir leading to enhancement in oil recovery. The population injected is typically in a fluid, such as the medium used to grow the microorganisms. The oil reservoir is also injected either concurrently with, or following injection of the microbial population, with a nutrient solution. The nutrient solution is one that is supportive of growth of the Shewanella cells in the population, and of the Arcobacter cells if present. The nutrient solution contains at least one carbon source and an electron acceptor. In some embodiments the electron acceptor is an organic electron acceptor or a metal ion electron acceptor as described above. In some embodiments the carbon source is acetate, lactate, succinate, butyrate or formate. In addition the nutrient solution may contain media components as described above.

The microbial population may be introduced into an oil reservoir by any method known to one of skill in the art. Typically introduction into an oil reservoir is by injecting into an injection well, but injection may also be into a production well. The injected fluids flow through the well and into the subterranean sites adjacent to the well. After introduction a period of time is allowed for growth of the introduced microorganisms. This period of microbial growth may be a week or more. In one embodiment this period is about two to three weeks. Following this period, injection water is introduced into the well bore and it flow through the well and into the subterranean sites adjacent to the well. However, now permeable rock is populated by the microorganisms so that the water displaces the oil in the oil reservoir. The water containing oil is recovered from at least one production well.

### General Methods

The meaning of abbreviations are used in this application are as follows: “hr” means hour(s), “min” means minute(s), “day” means day(s), “mL” means milliliters, “mg/mL” means milligram per milliliter, “L” means liters, “μL” means microliters, “mM” means millimolar, “μM” means micromolar, “nM” means nano molar, “μg/L” means microgram per liter, “pmol” means picomol(s), “° C.” means degrees Centigrade, “° F.” means degrees Fahrenheit, “bp” means base pair, “bps” means base pairs, “mm” means millimeter, “ppm” means part per million, “ppt” means part per thousand, “g/L” means gram per liter, “mL/min” means milliliter per minute, “mL/hr” means milliliter per hour, “cfu/mL” means colony forming units per milliliter, “g” means gram, “mg/L” means milligram per liter, “Kev” means kilo or thousands of electron volts, “psig” means per square inch per gram, “LB” means Luria broth, “rpm” means revolution per minute, “NIC” means non inoculated control.

### Growth of Microorganisms

Techniques for growth and maintenance of anaerobic cultures are described in “Isolation of Biotechnological Organisms from Nature”, (Labeda, D. P. ed. 117-140, McGraw-Hill Publishers, 1990). Fumarate is utilized as the primary electron acceptor under the growth conditions used herein. Nitrate was used in some enrichment as alternative electron acceptor control. Under denitrification, anaerobic growth is measured by nitrate depletion from the growth medium over time. The reduction of nitrate to nitrogen has been previously described (Moreno-Vivian, C., et al., J. Bacteriol., 181, 6573-6584, 1999). In some cases nitrate reduction processes lead to nitrite accumulation which is subsequently further reduced to nitrogen. Hence, accumulation and sometimes dissipation of nitrite is therefore also considered evidence for active growth and metabolism by microorganisms. Increase in turbidly due to increase in concentration of bacteria cells and/or the formation of biofilm on the bottom of the anaerobic serum vials or at their aqueous and gas interface were also taken as indicators of microbial growth.

### Ion Chromatography

To quantitate nitrate and nitrite ions in aqueous media, Applicants used an ICS2000 chromatography unit (Dionex, Banockburn, Ill.). Ion exchange was accomplished on an AS15 anion exchange column using a gradient of 2 to 50 mM potassium hydroxide. Standard curves using known amounts of sodium nitrite or sodium nitrate solutions were generated and used for calibrating nitrate and nitrite concentrations.

### Measurement of Total Dissolved Salts by Refractometer

The total dissolved salt was measured using a hand-held refractometer (Model RHS 10ATC, Huake Instrument Co., Ltd).

Samples from Oil Reservoir Production and Infection Waters

Petroleum well systems were sampled for this study. One is called Well system #1, which is in the Senlac field, located in the province of Saskatchewan, Canada and the other is called Well system #2, which is located in the Wainwright field in the province of Alberta, Canada. Resevoir water taken from Well system #1 has a salinity near Sea Water, which is in the range of 30 to 38 ppt and Well system #2 has a salinity of about twice seawater, which is in the range of 60 to 68 ppt. Water samples were obtained from production and injection well heads as mixed oil/water liquids in glass 1.0 L brown bottles, filled to the top, capped and sealed with tape to prevent gas leakage. Gas from inherent anaerobic processes sufficed to maintain anaerobic conditions during shipment. The bottles were shipped in large plastic coolers filled with ice blocks to the research facilities and arrived within 48 hr of sampling.

Genomic DNA Extractions from Bacterial Cultures

To extract genomic DNA from reservoir production and injection waters and liquid bacterial enrichment cultures, cells were harvested and concentrated by filtration onto a 0.2 micron Supor® Filter (Pall Corp, Ann Arbor, Mich.). An aliquot (2-5 mL) of a bacterial culture was passed through a 0.2 micron, 25 mm filter disk in a removable cartridge holder using either vacuum or syringe pressure. The filters were removed and placed in lysis buffer (100 mM Tris-HCL, 50 mM NaCl, 50 mM EDTA, pH8.0) followed by agitation using a Vortex mixer.

Reagents were then added to a final concentration of 2.0 mg/mL lysozyme, 10 mg/mL SDS, and 10 mg/mL Sarkosyl to lyse the cells. After further mixing with a Vortex mixer, 0.1 mg/mL RNase and 0.1 mg/mL Proteinase K were added to remove the RNA and protein contaminants and the mixture was incubated at 37° C. for 1.0-2.0 hr. Post incubation, the filters were removed and samples were extracted twice with an equal volume of phenol:chloroform:isoamyl alcohol (25:24:1, v/v/v) and once with chloroform:isoamyl alcohol (24:1, v/v). One-tenth volume of 5.0 M NaCl and two volumes of 100% ethanol were added to the aqueous layer and mixed. The tubes were frozen at −20° C. overnight and then centrifuged at 15,000×g for 30 min at room temperature to pellet chromosomal DNA. The pellets were washed once with 70% ethanol, centrifuged at 15,000×g for 10 min, dried, resuspended in 100 μL of de-ionized water and stored at −20° C. An aliquot of the extracted DNA was analyzed on an agarose gel to ascertain the quantity and quality of the extracted DNA.

Population Analysis of the Microorganism Populations Using rDNA Profiling Cloned Microbial Genomic 16S rDNA Libraries.

Genomic 16S rDNA libraries were generated from the petroleum reservoir production and injection waters samples from both well systems and from samples from enrichment cultures. Primer sets were chosen from Lane, ((1991) 16S/23S rRNA sequencing, p. 115-175. In Stackebrandt, E. and M. Goodfellow (ed.), Nucleic Acid Techniques in Bacterial Systematics. John Wiley and Sons, New York, N.Y.) to generate PCR amplified 16S rDNA fragments from microbial species present in the DNA samples prepared from the well samples or the enrichment cultures. The combination of forward primer (SEQ ID NO:1) and reverse primers (SEQ ID NO:2) were chosen to specifically amplify the bacterial 16S rDNA sequences.

The PCR amplification mix included: 1.0× Go Taq® PCR buffer (Promega), 0.25 mM dNTPs, 25 pmol of each primer, in a 50 μL reaction volume. 0.5 μL of Go Taq® DNA polymerase (Promega) and 1.0 μL (20 ng) of sample DNA were added. The PCR reaction thermal cycling protocol used was 5.0 min at 95° C. followed by 30 cycles of: 1.5 min at 95° C., 1.5 min at 53° C., 2.5 min at 72° C. and final extension for 8 min at 72° C. in a Applied Biosystems® GeneAmp® 9700 thermocycler (LifeTechnologies, Corp, Grand Island, N.Y.). This protocol was also used with cells from either purified colonies or mixed species from enrichment cultures.

The 1400 base pair amplification products for a given DNA pool were visualized on 0.8% agarose gels. The PCR reaction mix was used directly for cloning into pCR®-TOPO4® vector using the TOPO® TA cloning system (Invitrogen™, LifeTenchnologies, Corp, Carlsbad, Calif.) as recommended by the manufacturer. DNA was transformed into TOP10® chemically competent cells selecting for ampicillin resistance. Individual colonies (˜48-96 colonies) were selected and grown in microtiter plates for sequence analysis.

### Plasmid Template Preparation

Large-scale automated template purification systems used Solid Phase Reversible Immobilization (SPRI®, Agencourt, Beverly, Mass.; DeAngelis, M. M., et al., Nucleic Acid Res., 23: 4742-4743, 1995). The SPRI® technology uses carboxylate-coated, iron-core, paramagnetic particles to capture DNA of a desired fragment length based on tuned buffering conditions. Once the desired DNA is captured on the particles, they can be magnetically concentrated and separated so that contaminants can be washed away.

The plasmid templates were purified using a streamlined SprintPrep™ SPRI protocol (Agencourt). This procedure harvests plasmid DNA directly from lysed bacterial cultures by trapping both plasmid and genomic DNA to the functionalized bead particles and selectively eluting only the plasmid DNA. Briefly, the purification procedure involves addition of alkaline lysis buffer (containing RNase A) to the bacterial culture, addition of alcohol based precipitation reagent including paramagnetic particles, separation of the magnetic particles using custom ring based magnetic separator plates, 5× washing of beads with 70% ETOH, and elution of the plasmid DNA with water.

rDNA Sequencing, Clone Assembly and Phylogenetic DNA Analysis

DNA templates were sequenced in a 384-well format using BigDye® Version 3.1 reactions on ABI®3730 instruments (Applied Biosystems®, Foster City, Calif.). Thermal cycling was performed using a 384-well thermalcycler. Sequencing reactions were purified using Agencourt's CleanSeq® dye-terminator removal kit as recommended by the manufacturer. The reactions were analyzed with a model ABI3730XL capillary sequencer using an extended run module developed at Agencourt. All sequence analyses and calls were processed using Phred base calling software (Ewing et al., Genome Res., 8: 175-185, 1998) and constantly monitored against quality metrics.

Assembly of rDNA Clones

A file for each rDNA clone was generated. The assembly of the sequence data generated for the rDNA clones was performed by the PHRAP assembly program (Ewing, et al., supra). Consensus sequence and consensus quality files were generated for greater than one overlapping sequence read.

Analysis of rDNA Sequences

Each assembled sequence was compared to the NCBI (rDNA database; ˜260,000 rDNA sequences) using the BLAST® algorithm program (Altschul, supra). The BLAST® hits were used to group the sequences into homology clusters, each containing sequences with ≧90% identity to the same NCBI rDNA fragment. The nucleotide sequence of the 16S rDNA used to identify the Shewanella homology cluster was SEQ ID NO:3. The nucleotide sequence of the 16S rDNA used to identify the Arcobacter homology cluster was SEQ ID NO:4. The homology clusters were used to calculate proportions of particular species in any sample. Because amplification and cloning protocols were identical for analysis of each sample, the proportions could be compared from sample to sample. This allowed comparisons of population differences in samples taken for different enrichment selections or at different sampling times for the same enrichment consortium culture.

### DNA Preparation Bacterial Colony Isolates for Sequence Analysis

Genomic DNA from bacterial colonies plated on Marine agar was isolated by diluting bacterial colonies in 50 82 L of water or Tris-HCL buffer pH7-8. The diluted colony DNAs were amplified with Phi 29 DNA polymerase prior to sequencing (GenomiPHI Amplification Kit GE Life Sciences, New Brunswick, N.J.). An aliquot (1.0 4) of a diluted colony was added to 9.0 μof the Lysis Reagent (from the GenomiPHI® Amplification Kit) and heated to 95° C. for 3 min followed by immediate cooling to 4° C. 9.0 μL of enzyme buffer and 1.0 μL of Phi 29 enzyme were added to each lysed sample followed by incubation at 30° C. for 18 hr. The polymerase was inactivated by heating to 65° C. for 10 min followed by cooling to 4° C.

### DNA Sequence Analyses

DNA sequencing reactions were set up as follows: 8.0 μL of GenomiPHI amplified sample were added to 8.0 μL of BigDye® v3.1 Sequencing reagent (Applied Biosystems™, Foster City, Calif.) followed by 3.0 μL of 10 μM primers SEQ ID NOs:1, 2, 3, or 4 (prepared by Sigma Genosys, Woodlands, Tex.), 4.0 μL of 5× BigDye® Dilution buffer (Applied Biosystem™) and 17 μL Molecular Biology Grade water (Mediatech, Inc., Herndon, Va.).

Sequencing reactions were heated for 3.0 min at 96° C. followed by 200 thermocycles of (95° C. for 30 sec; 55° C. for 20 sec; 60° C. for 2 min) and stored at 4° C. Unincorporated dNTPs were removed using Edge Biosystems (Gaithersburg, Md.) clean-up plates. Amplified reactions were pipetted into one well of a pre-spun 96 well clean up plate. The plate was centrifuged for 5.0 min at 5,000×g in a Sorvall RT-7® (Sorvall®, Thermo Scientific®, Newtown, Conn.) at 25° C. The cleaned up reactions were placed directly onto an Applied Biosystems™ 3730 DNA sequencer and sequenced with automatic base-calling.

Each of the assembled rDNA sequences was compared to the NCBI rDNA database (260,000 rDNA sequences) using the BLAST® algorithm (Altschul et al., Journal of Molecular Biology, 1990). The highest scoring sequence identity hit was used as an identifier of the most closely related known species for strain identification.

Alternatively, to generate amplified rDNA fragments from individual strains, we chose the same primer sets from Lane, D. J. (supra). The combination of primer SEQ ID NO:1 and primer SEQ ID NO:2 was chosen to specifically amplify bacterial rDNA sequences.

The PCR amplification mix included: 1.0× GoTaq® PCR buffer (Promega), 0.25 mM dNTPs, 25 pmol of each primer, in a 50 μL reaction volume. 0.5 μL of GoTaq® DNA polymerase (Promega) and 1.04 (20 ng) of sample DNA were added. PCR reaction thermocycling protocol was 5.0 min at 95° C. followed by 30 cycles of: 1.5 min at 95° C., 1.5 min at 53° C., 2.5 min at 72° C. and final extension for 8 min at 72° C. in an Applied Biosystems™ GeneAmp® 9700 thermocycler. The 1400 base pair amplification products were visualized on 1.0% agarose gels. Sequencing of the amplified fragments and strain identification was as described above.

### EXAMPLE 1

### Anaerobic Enrichment for Indigenous Microbes from High Salinity Oil Reservoir Samples

Three different electron acceptors were used in oil reservoir sample enrichments. 1 mL of either injection water or production water from Well system #1 or #2, described in General Methods, was inoculated into 9 mL of minimal salts medium (Table 1) in 20 mL anaerobic serum vials. In addition, the minimal salts medium was supplemented with 43 ppt sodium chloride to bring its salinity to 53 ppt. The growth medium was then supplemented with 2000 ppm sodium lactate to serve as the carbon source and 3.7 g/L sodium fumarate as electron acceptor. Separate cultures were supplemented with 1600 ppm sodium nitrate or 2500 ppm Fe(III) added as ethylenediaminetetraacetic acid ferric sodium salt (EDTA (C₁₀H₁₂FeN₂NaO₈; E6760-100G , Sigma-Aldrich, St. Louis, Mo.) in place of fumarate. The medium was deoxygenated (anaerobic) by sparging the filled vials with a mixture of nitrogen and carbon dioxide (80%/20%) followed by autoclaving. All manipulations of the oil field water samples, which contained indigenous microbes from the reservoir, were performed in an anaerobic chamber (Coy Laboratories Products, Inc., Grass Lake, Mich.). The inoculated culture bottles were incubated at ambient temperature.

TABLE 1
Minimal salts medium

| g/L | Chemical |
|---|---|
| 1.0 | NH₄Cl |
| 0.5 | KH₂PO₄ |
| 0.4 | MgCl₂•6H₂O |
| 0.2 | CaCL₂•2H₂O |
| 10 | NaCl |
| 0.69 | NaH₂PO₄ |
| 2.5 | NaHCO₃ |
| 0.073 | KSO₄ |
| 1000X g/L | |
| Master mix | Trace elements |
| 1.5 | FeCl₂•4H₂O |
| 0.002 | CuCl₂•2H₂O |
| 0.1 | MnCL₂•4H₂O |
| 0.19 | CoCl₂•6H₂O |
| 0.07 | ZnCl₂ |
| 0.006 | H₃BO₃ |
| 0.036 | Na₂MoO₄•2H₂O |
| 0.024 | NiCl₂•6H₂O |
| 0.277 | HCl |
| 1000X g/L | Selenium/ |
| Master mix | tungstate |
| 0.006 | Na₂SeO₃•5H₂O |
| 0.008 | Na₂WO₄•2H₂O |
| 0.5 | NaOH |
| 1000X g/L | |
| Master mix | Vitamin mix |
| 100 | vitamin B12 |
| 80 | p-aminobenzoic |
| | acid |
| 20 | D(+)-Biotin |
| 200 | nicotinic acid |
| 100 | calcium |
| | pantothenate |
| 300 | pyridoxine |
| | hydrochloride |
| 200 | thiamine-HCL•2H₂O |
| 50 | Alpha-lipoic acid |

The pH of the medium was adjusted to 6.8, approximate reservoir pH.

The enrichments cultures containing nitrate were monitored and sampled regularly for nitrate depletion and nitrite accumulation, or in some cases, nitrite depletion. When nitrate was depleted in the sample (usually by 14 days), lactate and nitrate were added to the original final concentrations. At the same time, lactate and fumarate were added in each of the other enrichments to the original final concentrations as well.

All vials were incubated for an additional 14 to 20 days at room temperature. Visible changes in the cultures, which indicated growth on lactate and the electron acceptor, were observed in each of the enrichment samples. Changes included visible turbidity in the medium, and/or the presence of biofilms on the glass vials or at the gas-aqueous interface, as well as nitrate and nitrite reduction in vials containing nitrate as the electron acceptor. Turbidly was similar in each vial indicting that there was a diverse population of microorganisms in both the injection waters and the production waters.

### EXAMPLE 2

### Analysis of Populations in Anaerobic Enrichment Cultures

Before the enrichment cultures were started, the baseline distributions of the indigenous microbial populations in Well system #1 and Well system #2, were determined by rDNA population profiles, which were made using the reservoir production and injection waters from both systems using the method described above in General Methods. After 4 weeks of growth, all enrichment cultures were profiled using rDNA sequence distributions, as described in General Methods, to see differences in population profiles as compared to the indigenous populations.

Results of 16S rDNA Sequence Analysis for Well System #1

For Well system #1, 16S rDNA profiles were compiled for the production water and for a sample of the production water enrichment cultures. The homology clusters, each with 90% identity to the same NCBI rDNA fragment sequence file as described in General Methods, obtained for the parent production water sample for the fumarate enrichment culture, and for the Fe(III) enrichment culture were used to calculate the proportions of different types of bacteria in each sample. The results are given in Table 2. The nitrate enrichment culture was not evaluated in this sample.

TABLE 2
Phylogenetic Distributions in Well System#1 Production
Water and enrichment cultures

| | Proportion of population | | |
|---|---|---|---|
| Bacterial | Well System #1 | Fumarate | Fe(III) |
| identification | Production water | enrichment | enrichment |
| Epsilonproteobacter; | 71.7% | 65.2% | 10% |
| Arcobacter | | | |
| Gammaproteobacter; | nd* | 2.1% | 17.4% |
| Shewanella | | | |
| Other | 28.3% | 32.7% | 72.6% |

nd* means not detected

The results showed that bacteria belonging to the Arcobacter genus of the Epsilonproteobacteria class of Proteobacteria were the predominant type of bacteria present in Well System #1 production water (about 72%), and with fumarate enrichment the Arcobacter genus remained the predominant type of bacteria present with about 65% of the population. Also with fumarate enrichment the percentage of bacteria belonging to the Shewanella genus of the Gammaproteobacteria class increased to about 2% from being undetectable.

With Fe(III) enrichment the percentage of bacteria belonging to Shewanella increased, while the percentage of bacteria belonging to Arcobacter decreased.

The fumarate enrichment culture was incubated for another 14 days at room temperature, a 100 μL aliquot was streaked from the enrichment onto a Marine broth agar plate (made per recipe, Difco™ 2216, Becton-Dickenson, Sparks, Md.) and the plate was incubated at room temperature for two days. Marine broth has salinity of approximately 34 ppm (with 19.5 ppm NaCl). Electron acceptors in marine broth include sulfate, nitrate and unspecified organic electron acceptors from peptone and yeast extract.

Random colonies were restreaked onto Marine broth agar plates and grown to purify isolates. To characterized the identity of the isolates, the colonies were screened for identification by PCR amplification using direct colony rDNA analysis described in the General Methods using both the Lane reverse (supra) PCR primer 1492R (SEQ ID NO:1) and forward PCR primer 8F (SEQ ID NO:2). The DNA sequencing and analysis described in General Methods was used to obtain 16S rDNA sequences for microbial identification.

Bacteria belonging to the Shewanella genus were the predominant isolated colonies form the fumarate enrichment followed by streaking on solid Marine broth agar plates. Following this process, about 52% of the population was classified as Shewanella. Arcobacter cells were not isolated using this particular procedure.

The reduced salt concentration in the marine broth may have favored growth of Shewanella and other bacterial genera over growth of Arcobacter.

TABLE 3
Phylogenetic Distribution of Isolated Colonies
grown on Marinebroth Agar following Fumarate
Enrichment of Well System#1 Production Water

| Bacterial identification | Proportion of population |
|---|---|
| Epsilonproteobacter; | nd* |
| Arcobacter | |
| Gammaproteobacter; | 51.7% |
| Shewanella | |
| Other | 48.3% |

nd* means not detected

Results of 16S rDNA Sequence Analysis for Well System #2

For Well system #2 production and injection water samples, enrichment cultures were prepared as described above with fumarate, nitrate, or Fe(III) as the electron acceptor. Population profiles based on16S rDNA sequences, obtained as described in General Methods, were compiled for the production water, injection water, and enrichment cultures that are shown in Tables 4 and 5.

In the Well system #2 production water, Shewanella cells were not detected and Arcobacteria cells were not well represented, at 4% (Table 4). In both the fumarate and nitrate enrichments the proportion of bacteria belonging to Arcobacter increased to become the predominant type: about 89% and 85% of the population, respectively. The Arcobacter population had a small increase with Fe(III) enrichment. With fumarate and Fe(III) enrichments, the Shewanella population increased, while in the nitrate enrichment Shewanella cells were still not detected.

TABLE 4
Phylogenetic Distribution in Well System
#2 Production Water and its Enrichments

| | Proportion of population | | | |
|---|---|---|---|---|
| | Well System | Fumarate | Fe(III) | Nitrate |
| Bacterial | #2 Production | enrich- | enrich- | enrich- |
| identification | water | ment | ment | ment |
| Epsilonproteobacter; | 4% | 88.6% | 8.3% | 85.2% |
| Arcobacter | | | | |
| Gammaproteobacter; | nd* | 2.1 | 10.7% | nd |
| Shewanella | | | | |
| Other | 96.0% | 9.3% | 81% | 14.8% |

nd* means not detected

In Well system #2 injection water, Shewanella cells were again not represented, while Arcobacter cells were the predominant microorganisms. In the fumarate enrichment culture, the proportion of Shewanella cells increased from undetectable to 21% of the population. The proportion of Arcobacter cells decreased, but remained the most predominant species at 26%. In the Fe(III) enrichment the proportion of Arcobacter cells decreased to not detectable, while the proportion of Shewanella cells increased to about 90%. In the nitrate enrichment Shewanella cells remained undetected while Arcobacter cells were reduced, but were well-represented at about 22%.

TABLE 5
Phylogenetic Distribution in Fumarate Enrichment
of Well System #2 Injection Water

| | Proportion of population | | | |
|---|---|---|---|---|
| | Well System | Fumarate | Fe(III) | Nitrate |
| Bacterial | #2 injection | enrich- | enrich- | enrich- |
| identification | water | ment | ment | ment |
| Epsilonproteobacter; | 88.0% | 26.0% | nd* | 22.2% |
| Arcobacter | | | | |
| Gammaproteobacter; | nd | 21% | 90.4% | nd |
| Shewanella | | | | |
| Other | 12% | 53% | 9.6% | 77.8% |

nd* means not detected

### EXAMPLE 3

### Salt Tolerance of Shewanella

A Shewanella strain isolated from the fumarate enrichment of Well #2 injection water was grown at 32° C. in 1% Bacto tryptone medium containing varying amounts of NaCl. Actual salinity was approximately 10 ppt higher than the ppt of NaCl due to the contribution of the tryptone in the medium, as measured by actual refractometric readings. The growth rate was determined for the strain in each NaCl concentration by measuring OD600, and is shown in Table 6, where Growth rate k=log2/T, where T=doubling time.

TABLE 6
Growth rates of Well #2 Shewanella
strain in different abounts of NaCl

| | NaCl ppt | | | | | | | | |
|---|---|---|---|---|---|---|---|---|---|
| | 1 | 5 | 10 | 15 | 20 | 25 | 30 | 40 | 50 |
| Growth | 0.49 | 1.20 | 1.12 | 0.94 | 0.96 | 0.63 | 0.55 | 0.60 | 0.42 |
| rate | | | | | | | | | |
| (h⁻¹) | | | | | | | | | |

The isolated Shewanella strain grew best in a salinity of about 15 ppt (5 ppt of NaCl+10 ppt of tryptone), with declining growth rate at higher salinities. The growth rate was substantially reduced at salinities of 11 and 60 ppt.

What is claimed is:

1. A method for enriching a microbial population comprising:
a) providing an environmental sample from an oil reservoir fluid comprising an indigenous microbial population; and
b) growing said sample using medium comprising salt, at least one carbon source, and an electron acceptor selected from organic electron acceptors and metal ion electron acceptors to obtain an enriched microbial population;
wherein the proportion of Shewanella cells in the enriched microbial population of (b) is greater than the proportion of Shewanella cells in the environmental sample of (a).

2. The method of claim 1 wherein the medium of (b) has salt concentration that is between about 10 ppt and 55 ppt.

3. The method of claim 1 further comprising a step after (b) of growing the enriched microbial population of (b) using medium having salt concentration that is between about 15 ppt and 35 ppt to obtain a second enriched microbial population.

4. The method of claim 1 wherein the electron acceptor is an organic electron acceptor and wherein Arcobacter cells are present in the enriched microbial population of (b).

5. The method of claim 4 wherein the salt concentration is at least about 40 ppt.

6. The method of claim 1 wherein the at least one carbon source of (b) is selected from the group consisting of acetate, lactate, pyruvate, butyrate, and formate.

7. A method for isolating a strain of the Shewanella genus comprising:
a) providing an environmental sample from an oil reservoir fluid comprising an indigenous microbial population;
b) growing said sample using medium comprising salt, at least one carbon source, and an electron acceptor selected from organic electron acceptors and metal ion electron acceptors to obtain an enriched microbial population;
c) growing the enriched microbial population of (b) on medium having salt concentration that is between about 10 ppt and 40 ppt to obtain isolated colonies; and
d) screening the isolated colonies of (c) by rDNA sequence analysis against known microbial rDNA sequences;
wherein at least one Shewanella strain is identified.

8. The method of claim 7 wherein the medium of (b) has salt concentration that is between about 10 ppt and 40 ppt.

9. The method of claim 1 or 7 wherein the metal ion is selected from the group consisting of Fe(III), Mn(IV), Cr(IV), and mixtures thereof.

10. The method of claim 1 or 7 wherein the organic electron acceptor is selected from the group consisting of fumarate, trimethylamine-N-oxide, dimethyl sulfoxide, pyruvate, glycine, and mixtures thereof.

11. A method for enhancing oil recovery from an oil reservoir comprising:
a) providing an enriched microbial population obtained by the method of claim 1 or 3;
b) introducing the enriched microbial population of (a) into an oil reservoir;
c) introducing a nutrient solution comprising at least one carbon source and at least one electron acceptor into said oil reservoir; and
d) recovering oil from the oil reservoir;
wherein growth of the enriched microbial population enhances oil recovery.


SEQUENCE LISTING (4 sequences)

SEQ ID NO: 1 - DNA, 20 bp, Artificial Sequence
  Primer
cggttaccttgttacgactt

SEQ ID NO: 2 - DNA, 20 bp, Artificial Sequence
  Primer
agagtttgatymtggctcag

SEQ ID NO: 3 - DNA, 1500 bp, Shewanella indica
ttgatcatggctcagattgaacgctggcggcaggcctaacacatgcaagtcgagcggtaacatttcaaaagcttgcttttgaagatgacgagcggcggacgggtgagtaatgcctgggaatttgcccatttgtgggggataacagttggaaacgactgctaataccgcatacgccctacgggggaaagcaggggaccttcgggccttgcgctgatggataagcccaggtgggattagctagtaggtgaggtaatggctcacctaggcaacgatccctagctggtctgagaggatgatcagccacactgggactgagacacggcccagactcctacgggaggcagcagtggggaatattgcacaatgggggaaaccctgatgcagccatgccgcgtgtgtgaagaaggccttcgggttgtaaagcactttcagcgaggaggaaagggtgtaagttaataccttacatctgtgacgttactcgcagaagaagcaccggctaactccgtgccagcagccgcggtaatacggagggtgcgagcgttaatcggaattactgggcgtaaagcgtgcgcaggcggtttgttaagcgagatgtgaaagccccgggctcaacctgggaaccgcatttcgaactggcaaactagagtcttgtagaggggggtagaattccaggtgtagcggtgaaatgcgtagagatctggaggaataccggtggcgaaggcggccccctggacaaagactgacgctcaggcacgaaagcgtggggagcaaacaggattagataccctggtagtccacgccgtaaacgatgtctactcggagtttggtgtcttgaacactgggctctcaagctaacgcattaagtagaccgcctggggagtacggccgcaaggttaaaactcaaatgaattgacgggggcccgcacaagcggtggagcatgtggtttaattcgatgcaacgcgaagaaccttacctactcttgacatccagagaactttccagagatggattggtgccttcgggaactctgagacaggtgctgcatggctgtcgtcagctcgtgttgtgaaatgttgggttaagtcccgcaacgagcgcaacccctatccttacttgccagcgggtaatgccgggaactttagggagactgccggtgataaaccggaggaaggtggggacgacgtcaagtcatcatggcccttacgagtagggctacacacgtgctacaatggtcggtacagagggttgcgaagccgcgaggtggagctaatctcataaagccggtcgtagtccggattggagtctgcaactcgactccatgaagtcggaatcgctagtaatcgtggatcagaatgccacggtgaatacgttcccgggccttgtacacaccgcccgtcacaccatgggagtgggctgcaccagaagtagatagcttaaccttcgggagggcgtttaccacggtgtggttcatgactggggtgaagtcgtaacaaggtaac

SEQ ID NO: 4 - DNA, 1426 bp, Arcobacter marinus
ctggctcagagtgaacgctggcggcgtgcttaacacatgcaagtcgaacgagaacgggattagcttgctaatctgtcagctaagtggcgcacgggtgagtaatatataggtaacgtgccttcaagagggggataacagatggaaacgtctgctaagaccccatatgcctttaatacaaaagtatgaaagggaaatatttatagcttgaagatcggcctgtacagtatcagatagttggtgaggtaatggctcaccaagtcaatgacgcttaactggtttgagaggatgatcagtcacactggaactgagacacggtccagactcctacgggaggcagcagtggggaatattgcacaatgggggaaaccctgatgcagcaacgccgcgtggaggatgacacatttcggtgcgtaaactccttttatataagaagataatgacggtattatatgaataagcaccggctaactccgtgccagcagccgcggtaatacggagggtgcaagcgttactcggaatcactgggcgtaaagagcgtgtaggcggatagataagtcagaagtgaaatccaatagcttaactattgaactgcttttgaaactgtctatctagagtatgggagaggtagatggaatttctggtgtaggggtaaaatccgtagagatcagaaggaataccgattgcgaaggcgatctactggaacataactgacgctgagacgcgaaagcgtggggagcaaacaggattagataccctggtagtccacgccctaaacgatgtacactagttgttgctatgctcgacatagcagtaatgcagttaacacattaagtgtaccgcctggggagtacggtcgcaagattaaaactcaaaggaatagacggggacccgcacaagcggtggagcatgtggtttaattcgacgatacgcgaagaaccttacctggtcttgacatagtaagaaccatttagagatagatgggtgtctgcttgcagaaacttatatacaggtgctgcacggctgtcgtcagctcgtgtcgtgagatgttgggttaagtcccgcaacgagcgcaaccctcgtcattagttgctaacacttcgggtgagaactctaatgagactgcctacgcaagtaggaggaaggtgaggacgacgtcaagtcatcatggcccttacgaccagggctacacacgtgctacaatggggtatacaaagagcagcgatacagtgatgtggagcaaatctaaaaaatacctcccagttcggattgtagtctgcaactcgactacatgaagttggaatcgctagtaatcgtagatcagcaatgctacggtgaatacgttcccgggtcttgtactcaccgcccgtcacaccatgggagttgatttcactcgaagcggggatgctaagatagctaccctcctcag